(19) United States
(12) Patent Application Publication
(10) Pub. No.: US 2021/0065895 A1
(43) Pub. Date: Mar. 4, 2021

(54) SECURE COMMUNICATION AND DATA PROCESSING FOR RELATIONSHIP MONITORING

(71) Applicant: LifeCouple, San Diego, CA (US)

(72) Inventor: Sean Rones, San Diego, CA (US)

(21) Appl. No.: 17/008,309

(22) Filed: Aug. 31, 2020

Related U.S. Application Data
(60) Provisional application No. 62/894,723, filed on Aug. 31, 2019.

(51) Int. Cl. G16H 40/67; G16H 20/30; H04L 12/26; G06F 11/34

(52) U.S. Cl. CPC G16H 40/67; G16H 20/30; G06F 11/3409; G06F 11/3495; H04L 43/08

(57) ABSTRACT

Methods, devices, and systems for tracking, measuring, and advising users' interactions with their partners in different aspects of their relationship are described. An example method for monitoring a relationship includes displaying, to a first user, one or more metrics associated with the relationship between the first user and a second user, transmitting, to a remote server, the one or more metrics, receiving, from the remote server, at least one message, wherein the remote server is operable to generate the at least one message based on the one or more metrics, and wherein the at least one message comprises an activity or an engagement for the first user and the second user, and displaying, upon a determination that the activity or the engagement has not been performed, a reminder message to the first user.

### CROSS-REFERENCE TO RELATED APPLICATION

This patent document claims priority to and benefit of U.S. Provisional Patent Application No. 62/894,723 filed Aug. 31, 2019. The entire content of the before-mentioned patent application is incorporated by reference as part of the disclosure of this patent document.

### TECHNICAL FIELD

This document generally relates to data processing, and more specifically, tracking the level of interaction between members in a relationship.

### BACKGROUND

An interpersonal relationship is a strong, deep, or close association or acquaintance between two or more people that may range in duration from brief to enduring. The context can vary from family or kinship relations, friendship, marriage, relations with associates, work, clubs, and neighborhoods. Interpersonal relationships thrive through equitable and reciprocal compromise, they form in the context of social, cultural and other influences. The scientific study of relationships distinguishes itself from anecdotal evidence or from pseudo-experts by basing conclusions on data and on objective analysis.

### SUMMARY

Research has shown that the success of human interpersonal relationships depends on complex interactions between a large number of variables including, but not limited to, personality, socioeconomic status, religion, appearance, ethnic background, energy level, education, interests, and culture. Matching services (e.g., online dating apps, job portals, etc.) have developed systems that analyze these variables to identify and match people who have the potential to establish a successful relationship, but offer no longitudinal results on effectiveness.

However, services that facilitate maintaining and nurturing relationships are few and far between. Embodiments of the disclosed technology can track, measure, and advise users' interactions with their partners in different aspects of their relationship, thereby providing a sense of accountability for all participants, and strengthening the relationship.

In an aspect, an example method for monitoring a relationship includes displaying, to a first user, one or more metrics associated with the relationship between the first user and a second user, transmitting, to a remote server, the one or more metrics, receiving, from the remote server, at least one message, wherein the remote server is operable to generate the at least one message based on the one or more metrics, and wherein the at least one message comprises an activity or an engagement for the first user and the second user, and displaying, upon a determination that the activity or the engagement has not been performed, a reminder message to the first user.

In another aspect, an example method for monitoring a relationship includes tracking, based on a mobile application being used by a first user and a second user, one or more metrics associated with the relationship between the first user and the second user, generating, based on the one or more metrics, at least one message, and transmitting the at least one message to the first user or the second user.

In yet another aspect, an example method for monitoring a relationship includes receiving, from a mobile application via a first secure connection or a remote server via a second secure connection, one or more metrics associated with the relationship between a first user and a second user, receiving, from the first user over the first secure connection, a first message, and transmitting, to the first user, a second message, wherein the second message is operable to strengthen the relationship and is based on the one or more metrics and the first message.

In yet another aspect, the above-described methods are embodied in the form of processor-executable code and stored in a computer-readable program medium.

In yet another aspect, a device that is configured or operable to perform the above-described methods is disclosed.

The above and other aspects and their implementations are described in greater detail in the drawings, the descriptions, and the claims.

### BRIEF DESCRIPTION OF THE DRAWINGS

FIG. 1 shows an example of a system for secure communication and data processing for relationship monitoring.

FIG. 2 shows an example of a framework for secure communication and data processing for relationship monitoring.

FIG. 3A shows an example of a secure communication framework used between three parties for relationship monitoring.

FIGS. 3B and 3C show example timelines for the secure communication framework described in FIG. 3A.

FIGS. 4A-4I show screenshots of an example of a UI for a mobile app.

FIGS. 5A and 5B show screenshots of another example of a UI for a mobile app.

FIGS. 6A-6F show screenshots of yet another example of a UI for a mobile app.

FIGS. 7A-7D show screenshots of yet another example of a UI for a mobile app.

FIG. 8 shows an example of server and mobile application operation for secure communication and data processing for relationship monitoring.

FIGS. 9A-9H show screenshots of an example of a UI for a web/analysis interface.

FIG. 10 shows a block diagram of an example device that can implement embodiments of the disclosed technology.

FIGS. 11-13 show examples of methods for monitoring a relationship.

### DETAILED DESCRIPTION

Interpersonal relationships are dynamic systems that change continuously during their existence. Like living organisms, relationships have a beginning, a lifespan, and an end. They tend to grow and improve gradually, as people get to know each other and become closer emotionally, or they gradually deteriorate as people drift apart, move on with their lives and form new relationships with others.

Good relationships require ongoing commitment and work. Embodiments of the disclosed technology enable couples to address and monitor any challenges in their relationship. In an example, this is achieved by generating methods designed to encourage people to look more closely at their relationship across four key areas: trust, communication, conflict and intimacy. The methods includes daily relationship challenges, ice breakers to help approach tricky conversations, digital gifts, and the like.

Section headings are used in the present document to improve readability of the description and do not in any way limit the discussion or the embodiments (and/or implementations) to the respective sections only.

### 1. Embodiments of the Relationship Monitoring System

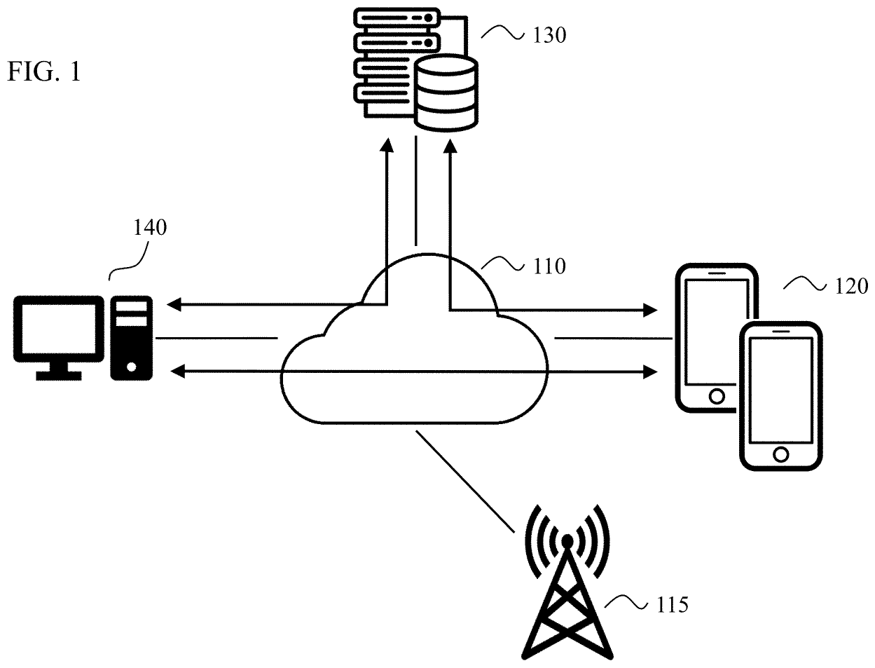

FIG. 1 shows an example of a system for secure communication and data processing for relationship monitoring. A shown therein, the system includes one or more mobile applications 120, a web/analysis interface 140 and a remote server 130, all connected to each other via a network 110. In some embodiments, the mobile application 120 may connected to the network via a cellular connection 115. In some embodiments, the system shown in FIG. 1 may be configured for a variety of difference scenarios or applications. In an example, the one or more mobile applications 120 may be partners in a relationship and the web/analysis interface 140 may be used by a therapist or counselor. In another example, the one or more mobile applications may be used by employees or members of a sports team, and the web/analysis interface 140 may be used by a human resources (HR) team member or a coach, respectively.

More generally, embodiments of the disclosed technology may be configured to enable the maintenance and/or nurturing of relationships, thereby promoting a sense of wellness in the users of the one or more mobile applications 120. In some embodiments, the participants using the system shown in FIG. 1 (e.g., via the web/analysis interface 140 and the one or more mobile applications 120) may further communicate with one or more financial services (e.g., an insurance company or financial advisor, not shown in FIG. 1), since finances and financial decisions are, too often, a very important part of a relationship.

### 1.1 Exemplary Applications

The following scenarios or frameworks are envisioned for the application of embodiment of the disclosed technology:
Tool for human resources (HR) and 360-degree reviews
Corporate team building
Team sports
Kids in varying familial situations (e.g., divorced parents, latch-key kid)
Mentoring relationships

### 1.1.1 Human Resources (HR) and 360° Review

In some embodiments, an employee downloads the mobile application 120. In an example, an initial step (e.g., an onboarding process) includes asking the employee questions that access vital areas that determine their satisfaction level in the following areas, which tie into the overall work/job satisfaction:
Appreciation
Compensation
Relationship with colleagues
Relationship with superiors (e.g., managers, supervisors)

In some embodiments, after the employee finishes the onboarding process, the remote server 130 will unlock portions of the mobile application 120 that provide content, activities and tools. In an example, the generation of these activities and content is based on machine learning (ML) and artificial intelligence (AI) algorithms, which enables the platform to offer dynamic user engagement. In another example, these activities and content may be gamified to challenge the employee to increase their productivity levels or to acquire new skills, and/or offer selections based on previous results in content effectiveness (e.g., previous results for that employee, the group or department that employee is in, etc.). These tools and activities will be offered and pushed via the HR department to the remote server 130 to then be pushed to the employee via the mobile application 120.

In some embodiments, the embodiments of the disclosed technology advantageously enable an employee's personal identification to remain unavailable to HR via web/analysis interface 140 or at all, thereby ensuring that any negative or critical dissatisfaction cannot be pin pointed on the employee level.

In some embodiments, the platform (e.g., one or more components of the system shown in FIG. 1) will enable employees in different departments to obtain a different user experience. For example, warehouse employees will be treated differently than marketing employees based on the remote server 130 employing machine learning and AI algorithms that understand the different personalities and motivation factors of employees in different departments.

In some embodiments, the HR department will be able to see a weekly, monthly and year-to-date (YTD) employee satisfaction levels and develop goal-oriented functions or activities to achieve higher job satisfaction levels.

In some embodiments, an employee may be using the platform both at work and for his/her personal relationship. In this scenario, and based on the amount of data the user is willing to share, the relationship attributes of the employee could be used to investigate how personal and family dynamics affect job performance.

In some embodiments, the remote server 130 will be able to anonymize the data, and compare it to that of other companies in similar sectors (and accounting for size, corporate structure, etc.), thereby becoming an accurate source for true corporate culture performance.

In some embodiments, the company founders or senior management will also be able to log in and examine reports, and further have the ability to compare the data to other key performance indications (KPIs) that determine company success goals. These computed scores for the employees will also be stored in the remote server 130, and can be leveraged if the employee signs up for other embodiments of the disclosed technology.

### 1.1.2 Team Sports

In some embodiments, a team member (or player) downloads the mobile application 120. In an example, an initial step (e.g., an onboarding process) includes asking the employee questions that access vital areas that determine their satisfaction level in the following areas, which tie into the team members satisfaction and integration into the team:
Team Morale
Coaching technique
Appreciation
Compensation

In some embodiments, after the team member finishes the onboarding process, the remote server 130 will unlock portions of the mobile application 120 that provide content, activities and tools. In an example, these activities and content may be gamified to challenge the employee to increase their productivity levels or to acquire new skills. These tools and activities will be offered and pushed via the player personnel department to the remote server 130 to then be pushed to the employee via the mobile application 120.

In some embodiments, the embodiments of the disclosed technology advantageously enable a team player's personal identification to remain unavailable to the personnel department, thereby ensuring that any negative or critical dissatisfaction cannot be pin pointed on the player-level.

In some embodiments, the platform will enable players in different positions to obtain a different user experience. For example, a lineman versus running back on a football team, a goalie vs a defense player on a soccer team, or an outfielder versus a pitcher on a baseball team, based on the remote server 130 employing machine learning and AI algorithms that understand the different personalities and motivation factors of team players in different positions on the team.

In some embodiments, the team personnel department will be able to see a weekly, monthly, and year-to-date (YTD) satisfaction levels of players via web/analysis interface 140 and develop goal-oriented functions or activities to achieve higher satisfaction and team unity levels. In other embodiments, a true locker room gauge can be created, which can also incorporate player statistics and performance based on the indices being monitored.

In some embodiments, the team owner or senior management will also be able to log in to web/analysis interface 140 and examine these reports, and further have the ability to compare them to other key performance indications (KPIs) that determine the team's success goals. These computed scores for the players will also be stored in the remote server 130, and can be leveraged if the employee signs up for other embodiments of the disclosed technology (e.g., a personal relationship in addition to the team membership).

In some embodiments, a player may be using the platform both as a member of the team and for his/her personal relationship. In this scenario, and based on the amount of data the user is willing to share, the relationship attributes of the employee could be used to develop correlations between player performance and personal/family dynamics.

In some embodiments, the remote server 130 will be able to compare the data to that of other teams, thereby becoming an accurate source for performance in a particular sport.

### 1.1.3 Kids and Family Dynamics

Embodiments of the disclosed technology can be adapted for use by parent and their children with regarding to monitoring family dynamics. Parents can download the mobile application 120. In an example, an initial step (e.g., an onboarding process) includes asking the parents questions that determine the satisfaction level of the dynamics in the family and their roles. The platform will also tie into their relationship satisfaction level, which has an integral role and ties into the family dynamics.

In some embodiments, each parent will have their own individual index level as well as an overall family dynamic score (e.g., based on both parents, which is a single score per family).

In some embodiments, the children will answer questions based on areas of satisfaction that the parents have selected, which will allow them to monitor vital areas for growth and improvement. In an example, a child profile will be associated with 4 key indexes that will have a score based on the dynamics of parent's relationship indexes, parent's satisfaction levels in areas of vital family dynamics, and satisfaction levels of the questions the child answered during the onboarding process. An example of indexes will can be tracked, and used to generate a score on overall family happiness and child happiness, include:
Accountability
Appreciation
Attitude
Respect

In some embodiments, the remote server 130 and mobile application 120 provide gamification methods to encourage the child and the parents to work towards increasing the key indexes that make up the overall family satisfaction level. For example, when the child completes a survey the child receives points and can use points to access other content. The indexes will communicate to the child in a way that increases optimal growth and will allow a parent to explain and monitor the factors that they have in this role.

In some embodiments, and since a child will not understand that parent's role and position, the parents will not be monitored by indexes of their performance. In contrast, they will be given activities, tools and content that will be tracked on their participation and engagement with the platform. Families will reach certain levels, which will unlock special offers that encourage them to interact with one another to improve their family dynamics.

Embodiments of the disclosed technology may be configured to identify specific situations, e.g., if the parents are divorced, a latchkey child, a single parent situation, varying economic situations, etc. The platform will consider all these aspects, and using machine learning and AI algorithms, find the most appropriate tools, content and techniques to address these scenarios and unique family dynamics (e.g., suggesting a fancy dinner for a family with modest means would be inappropriate).

In some embodiments, the remote server 130 and mobile application 120 will also be able to monitor factors like social media use, screen time use, and dynamics of friends and peers. For example, the parents will be able to track (and possibly limit) how much time their child(ren) spent on Facebook or Twitter. For another example, the parents can use the platform to coordinate playdates for their child(ren) with other children of the same age whose parents also use the platform. This can be initiated anonymously or through a common friend or coworker, which can be determined by the remote server 130. The platform may also support integration with family therapy and provide the therapist the ability to monitor the family dynamics prior to the first session, and also offer ways to assign relationship homework assignments and practice exercises in between sessions.

In some embodiments, the platform will also have the ability to monitor the medication prescribed to a child, as well as the child's reactions and moods, thereby providing the therapist and parents a gauge of the effect the medication is having to the child's overall well-being. For example, the user can be prompted to take their medication and confirm they have taken the medication. The mobile application 120 can ask the user to answer a questionnaire at various intervals or play a game that monitors reaction times. The mobile application 120 can use the camera to monitor pupil dilation or other visual symptoms relating to the medication, and or biometrics data the mobile application has access to. The mobile application 120 can prevent other activities on the phone until the user confirms they have taken their medication, responded to the questionnaire or played the game. In an example, these indices can determine the following:
Happiness
Energy Level
Focus
Friendships

These and other indices can help determine the efficacy of a drug in increasing or decreasing these indices, as well as what the child is being treated for. The platform will also be able to provide monitoring if a parent tries a natural approach (e.g., homeopathy), thereby changing the child's diet and other key factors. In an example, the platform can consolidate these findings across several families, and use machine learning and AI to offer correlations based on factors such as race, economic factors, family history, parent's relationship, parents' job satisfaction, family time, etc.

In some embodiments, and depending on the medication, smartphone activity and interactions with the other partners may be measured before and after the medication.

### 1.2 Examples of “Big Data” Processing

In some embodiments, the data collected by embodiments of the disclosed technology, discussed in more detail below, can be used to conduct cross-sectional and longitudinal studies. For an example, cross-sectional studies (analysis of data over a number of subjects and variables for a specific time) can be performed, and those results can be fed into longitudinal studies (analysis over time for a specific number of subjects and/or variables) to improve the remote server 130 algorithms. For another example, the analytical results may be provided to researchers and scientists, which will enable the prediction and treatment of diseases based on life factors.

In some embodiments, the data will be processed using a data science driven engine that is stored on the remote server 130, and have the ability to process the data after the mobile application 120 or the remote server 130 anonymizes the data. For example, overlaying data sets can be processed by machine learning and AI algorithms to create segments and clusters, which can then be used for A/B tests to then see how performance has changed. This can be iteratively performed on larger (and/or different) data subsets. This will advantageously enable the algorithms to be modified based on better and more refined data as time goes on. In an example, the mobile application 120 encourages involvement (e.g., sending text messages, pushing messages and emails) and, through gamification, rewards participants by offering third-party incentives and encourages the users to maintain their involvement. For an example, gamification rewards may be awarded based on maintaining a level of performance for a predetermine number of days (e.g., provide encouragement to your partner for N consecutive days). For another example, incentives may be awarded for engaging in different types of activities with one's partner.

Embodiments of the disclosed technology, in the various frameworks discussed above, strengthen relationships by addressing the mental, physical and emotional health of its participants. In some embodiments, the one or more mobile applications 120 may be configured to recognize when other participants are in its geographic vicinity. In other embodiments, the remote server 130 may implement geofencing, thereby triggering certain 3rd party applications and/or services based on the behavior of the participants in the relationship. For example, certain offers could be unlocked based on the proximity of the users to a specific location (e.g., movie or live event tickets, restaurant vouchers, etc.). For another example, offers could be unlocked to encouraging brand loyalty once a user's preferences have been ascertained. For another example, offers could be dynamically unlocked based on a combination of one or more relationship indexes (e.g., merging the scores, over overlaying specific indexes) of both partners or the entire family. These unlocked offers could be tracked and their use may unlock additional offers that promote the growth of the particular indexes that were overlaid.

Embodiments of the disclosed technology compile vital life attributes that theory-based experts have made reference to in the determination and development of their claims, and use data collected from a very large subject pool to help increase empowerment by using science. For example, indexes for different areas of a relationship tracked by the platform can be used to validate the efficacy of existing relationship satisfaction scales (e.g., the Locke-Wallace Marital Adjustment Test (MAT), the Quality of Marriage Index (QMI), the Relationship Assessment Scale (RAS), the Kansas Marital Satisfaction Scale (KMSS), etc.) by examining the specific criteria used by a particular scale and granular relationship data collected by the platform.

With this large data collection and interaction based on digital technology (e.g., the mobile phone, which people spend 4+ hours a day on), a phenomenology-based model can be developed using machine learning and AI algorithms, and may contribute to the common goal and desire to live a happy and empowered life. That is, the described embodiments and implementations are examples of relationship empowerment science, which provides a sense of accountability to individual persons in a relationship, thereby strengthening and maintaining the relationship.

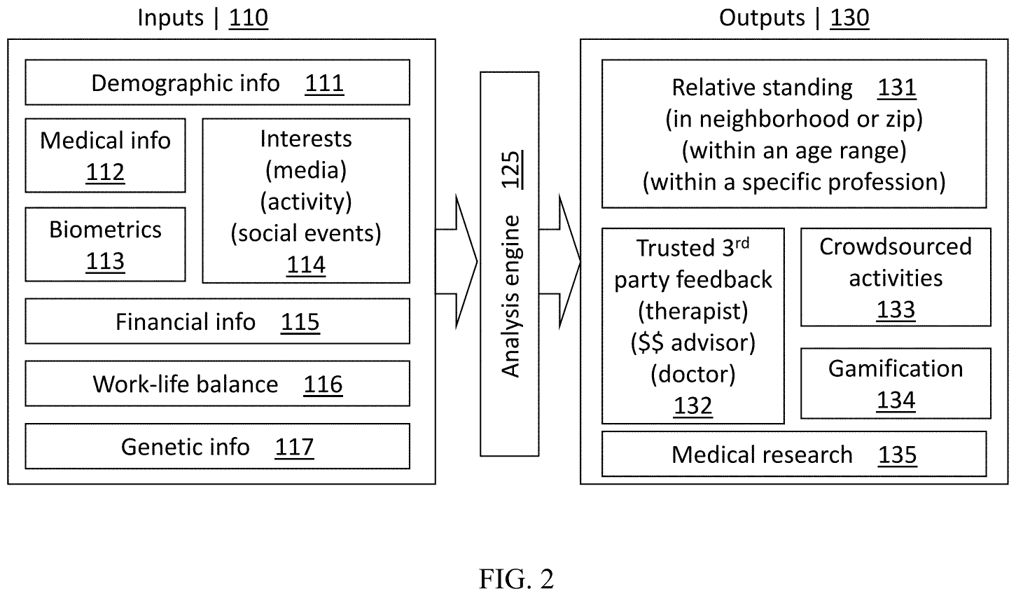

FIG. 2 shows an example of a framework for secure communication and data processing for relationship monitoring. As shown therein, an example system receives inputs 110 and processes them (via processor or analysis engine 125) to generate outputs 130. In an example, the inputs 110 may include demographic information 111 (e.g., age or gender), medical information 112 (e.g., current and past medication, sleep data), financial information 115 (e.g., savings rate), biometric information 113 (e.g., heart-rate) and/or genetic information 117 about the participant(s). The inputs may be received from users, a business organization, or a sports team. In another example, the inputs may further include a participant's interests 114, such as the current book they're reading, details of their media consumption, what social events have been or are planning to be attended, travel destinations and time spent on social media. The inputs may further include aspects and details of a participants work/life balance 116. The inputs shown in FIG. 2 are a non-exhaustive list of examples, and may further music and movie preferences, food and cuisine preferences, personality type and/or family history.

In some embodiments, these inputs are processed by the analysis engine 125 that generates one or more outputs 130. In an example, the outputs 130 include a participant's relative standing 131 (e.g., in their neighborhood or zip code, within the participants age range or their specific professor). In another example, the outputs 130 include crowdsourced activities 133 that the participant may enjoy and/or gamification 134 aspects. In yet another example, the outputs 130 may include feedback from trusted 3rd parties 132 (e.g., therapist, financial advisor, doctor). The outputs may be displayed on mobile application 120 or web/analysis interface 140.

In an example, outputting “a relative standing in a zip code” could include selecting which factors or metrics need to be displayed, ensuring enough data has been collected so output is statistically valid, making sure the data is anonymized, identifying any outliers in the data that should not be included, and aggregating across others with the same zip code to generate output.

In yet another example, the outputs 130 may include medical research 135. For example, the large data sets that may be collected by embodiments of the disclosed technology can be used to test anthropological or behavioral theories (e.g., why is the divorce rate higher than average in Spain, but significantly lower than average in Chile?).

In some embodiments, the data science driven engine (or analysis engine 125) may be configured to provide dynamic engagement opportunities (e.g., goals, incentives, advise, feedback, assurance and encouragement) for specific cohorts. For an example, the large data sets that can be collected may be specifically used for members of the armed services, the LGBTQ community, people of a certain faith, etc. For another example, the collected data may be used to provide content and assurances for users in specific stages/phases of their life (e.g., new job, new team, newly married, etc.).

### 1.2.1 A “Score” for Life and Happiness

In some embodiments, and especially if the platform is employed by a user to different aspects of their life (e.g., personal relationship, job, team sport, etc.), the integration of data across these aspects can be used to generate a “score” for a user's life or outlook, which can advantageously be used inform life decisions. For an example, this overall score may be used to find an ideal match in marriage or a second marriage, the type of company that is tailored to a user's strengths and interests, the type of parenting style that may be most effective and sustainable, the selection of next sports team to sign with, etc. For another example, the data could be used from the organization's standpoint to determine which employee to hire or which player to draft from the multitude of available choices.

More generally, partners can be matched via gauging their relationships and then integrating factors like job satisfaction, parenting dynamics or satisfaction. The platform may be configured to communicate quantifiable relationship data with the users, thereby encouraging them to track and monitor their progress and enabling them to take in consideration vital factors in one's life and create an accurate and evolving roadmap to the direction of empowerment.

### 1.2.2 Score Normalization

In some embodiments, the data collected may be used to normalize a user's score or values of one or more indices related to the relationship monitoring process. For an example, the normalization process may be used to determinate the “relative standing in a zip code” as described above. For another example, the third-party mediator may use normalization to better guide or assist a client by recognizing the inherent limitations in a particular scenario. Any relationship may be limited by resources and/or other external factors, and thus success in the relationship must be determined or gauged in light of these constraints. For example, if it is determined that the best that can be achieved on a certain index is 85/100, and if a score of 75/100 is achieved, then it may be a better source of motivation and reflective of the situation if a normalized score of 88/100 (75/85=88) is displayed.

In some embodiments, score normalization may be used when outputting results for specific life stages/phases for the user. A “relative standing” that is determined within a cohort may provide a more realistic gauge of success, especially for certain phases of life (e.g., job satisfaction in 20s and 30a, family dynamics in 40s and 50s, etc.)

Score normalization may be advantageously used to better align the interests of the organization (e.g., the team, family, relationship, company) in view of the capabilities of the members of that organization (e.g., players, partners, employees). In some embodiments, the normalized score may be determined based on moderated (or asymmetric) messaging, which is discussed in Section 4.1.

### 2. Examples of Secure Communication Enabling Monitoring Relationships

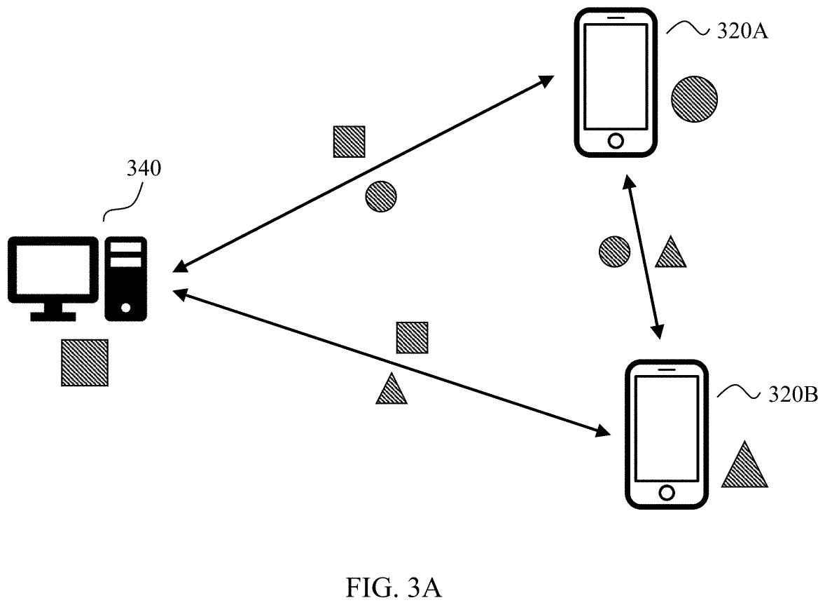

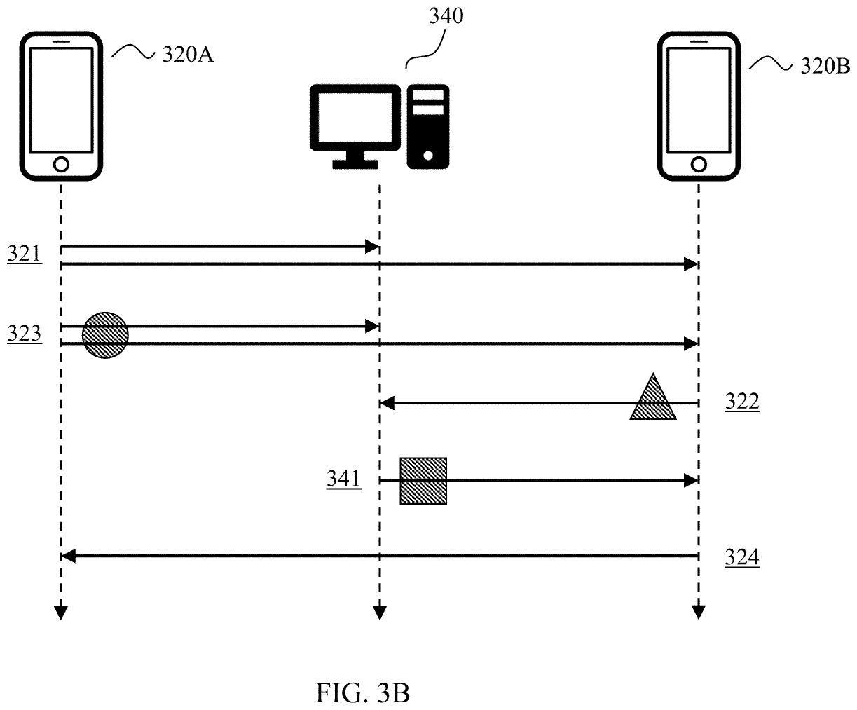

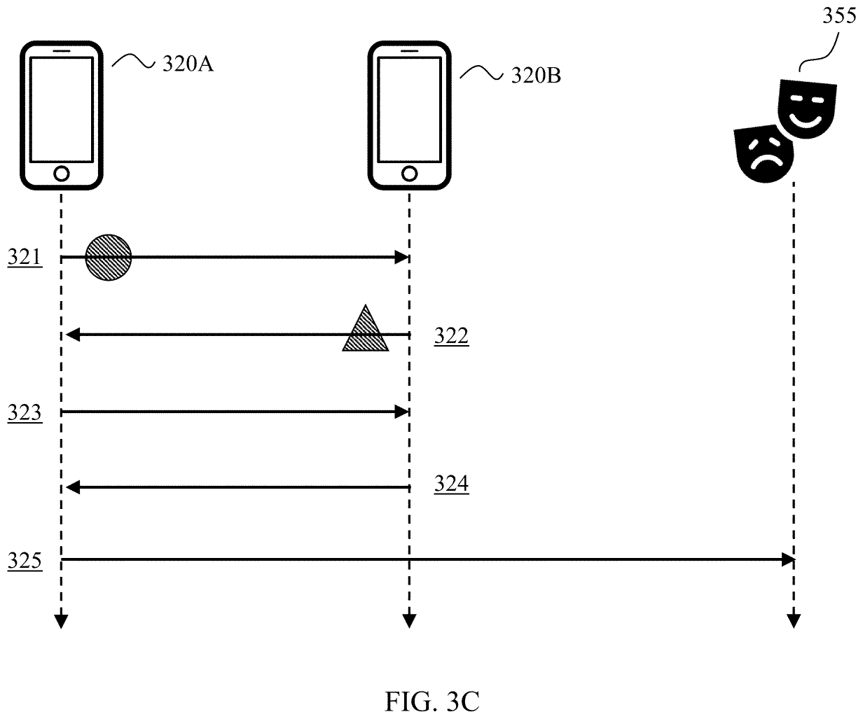

Data security and privacy are paramount in embodiments of the disclosed technology. FIG. 3A shows an example of a secure communication framework used between three parties for relationship monitoring, wherein each party (or user) has a unique token (represented as a circle for 320A, a triangle for 320B and a square for 340) that can be used to ensure private point-to-point communication. In some embodiments, the data generated and processed by the users (320A and 320B) and the third-party mediator (340), which may be sent through or stored on the remote server (not shown in FIG. 3A) may include “protected health information” (PHI), or more specifically, electronic PHI (ePHI). In some embodiments, ePHI is received, created, maintained and transmitted in compliance with the Health Insurance Portability and Accountability Act (HIPAA).

In some embodiments, and as shown in FIG. 3B, Partner A (320A) may be communicating with the therapist (340) and Partner B (320B). In an example, an initial unencrypted communication (321) may be an invite from Partner A to both the therapist and Partner B, and may be followed by a prior medical or psychological record (323) that is secured using an encryption technique that uses Partner A's token. Similarly, Partner B may exchange secure messages with the therapist (322 and 341), which are encrypted using their respective tokens. In an example, tokens may be implemented as public key (or asymmetric) cryptography with private keys being stored by the user and the public key being available to other parties.

In some embodiments, an as shown in FIG. 3C, secure messaging (321 and 322) between Partner A (320A) and Partner B (320B), may be followed by unencrypted messages that include communication (325) with a third-party provider 355. For example, as part of building the relationship, Partner A may initiate an activity (e.g., night at the theater) that can be discussed with Partner B (messages 321 and 322), and then organized (323, 324 and 325).

Embodiments of the disclosed technology, as part of HIPAA compliance, can implement one or more of physical safeguards, technical safeguards, technical policies and network (or transmission) security for the data/ePHI being received, created, maintained and transmitted in the examples described in this document.

The mobile application advantageously enables, based on the secure communication framework described in the context of FIGS. 3A-3C, the participant to control their data at various levels of granularity. In some embodiments, the mobile application may be used by one half of a couple 320A who can choose to share certain details with their significant other 320B, but different details with their therapist 340. FIG. 3C shows an example of secure messaging between a couple with the ability to contact external third parties (e.g., to plan an activity).

### 3. Embodiments of the Mobile Application and the Remote Server

Embodiments of the disclosed technology provide several algorithms and methods to monitor and foster relationships with an eye on accountability.

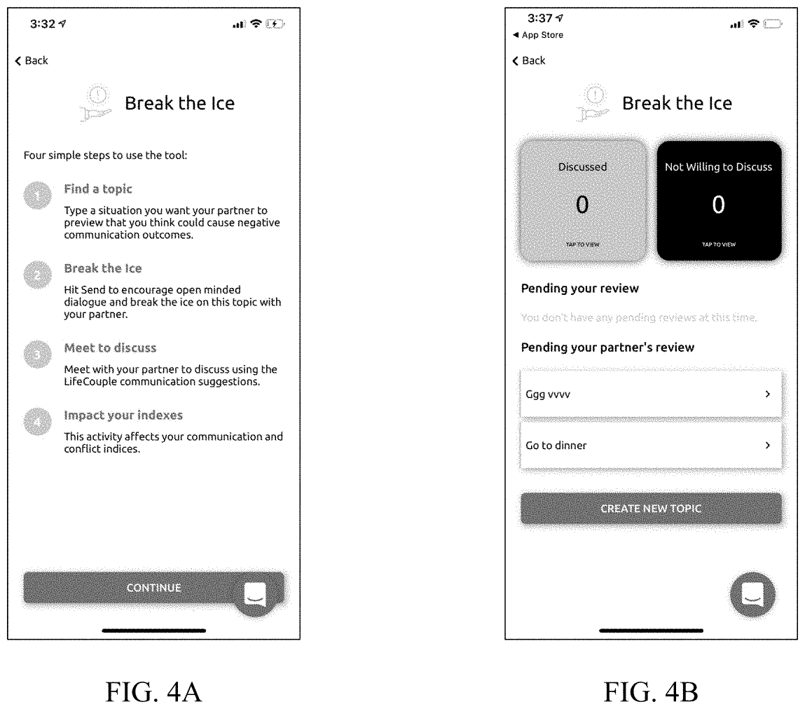

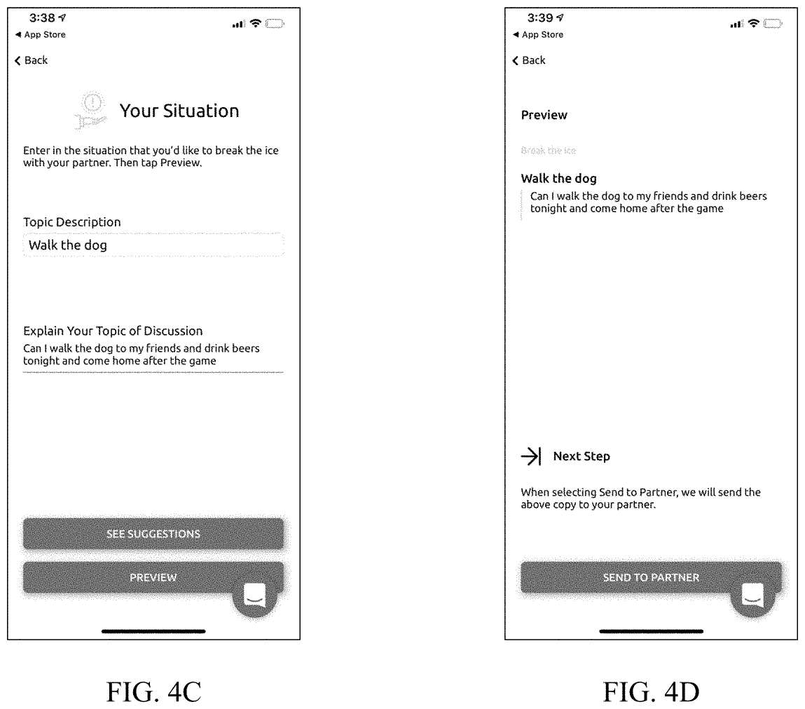

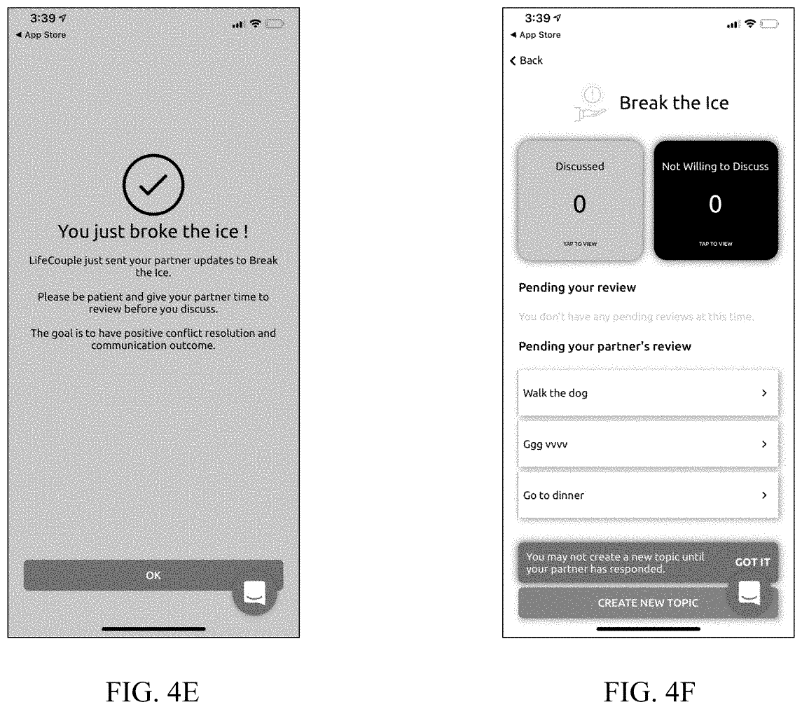

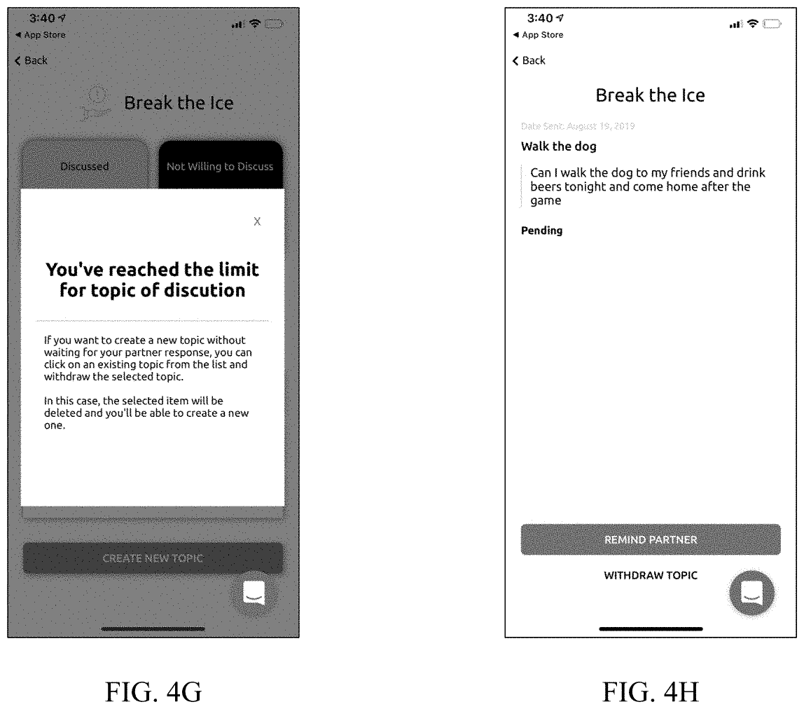

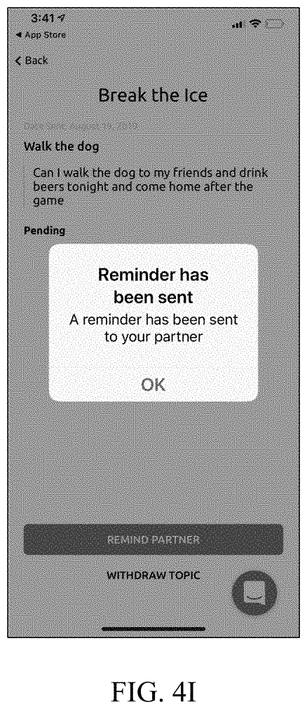

Breaking the Ice. This method is directed to dealing with tough or difficult topics in a relationship using the platform in conjunction with relationship techniques to promote healthy conflict resolution, and is illustrated in using the example screenshots in FIGS. 4A-4I. In an example involving Partners A and B, Partner A would identify a topic or situation (e.g., FIG. 4C and FIG. 4D) that has caused conflict in the relationship and describe the proposed actions or activities to alleviate this. The platform (e.g., the remote server) sends a message to the Partner B (e.g., FIG. 4E) explaining how important it is to try to meet and work through a particular situation with a positive result. The platform keeps track of what Partners A and B agreed to discuss and were not willing to discuss, and the partners are sent text messages. The messages include links that direct them to the topic, and facilitate a meeting, by encouraging them to select a date and time and meet. In effect, their interaction is monitored and they are encouraged to deal with the topic or situation in a different way in order to break the pattern. The platform then sends a message that asks if this was accomplished and asks via the Relationship Pulse check-in method, whether this was done with a level of satisfaction acceptable to both Partners A and B.

In some embodiments, the platform can track and determine whether one of the partners is having a more difficult time, and is also able to tie into a therapist evaluation of the dynamics between the two partners. That is, the platform serves as a proxy referee or score card to the actual situations being dealt with by Partners A and B, and mitigates (and sometimes even eliminates the finger pointing and deflecting of addressing issues).

In some embodiments, the platform also allows the person to withdraw the request, does not allow too many to pile up (e.g., FIG. 4G) so as to appear that one partner is barraging the other partner with constant issues. Additionally, the platform sends reminders (e.g., FIG. 4H and FIG. 4I) and messages in a fun and effective way to encourage positive relationship growth.

In some embodiments, and as discussed earlier, the platform also keeps track of scores and enables the gamification of the activities and discussions if it is appropriate for the situation.

In some embodiments, the Breaking the Ice method can be configured to recognize if a particular situation is toxic, and that a face-to-face meeting would be more harmful than helpful. In this case, the platform may be used to send an invite to both partners to attend a webinar or offer video and content on the importance of how that particular aspect should be dealt with in a relationship.

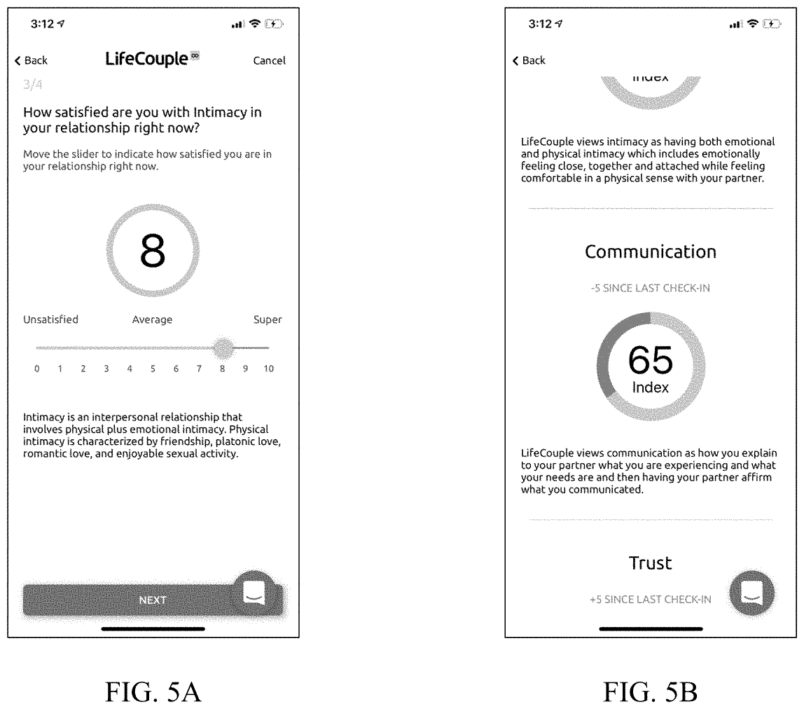

Relationship Pulse Check-In. This method is directed to gauging the current health of a relationship. In an example, the platform will randomly send a partner one or more of a text message, email message and push message request to check the health of their relationship, as shown in FIGS. 5A and 5B. This process will allow the platform to gauge any movement (or progress), either positive and negative. The platform will track this and offer a report to see the relationship history, and are the mechanism that updates the indexes and allows the couples to see progress of work towards a common goal of increasing the health of their relationship.

In some embodiments, the questions in the Relationship Pulse Check-In are dynamically generated in the remote server based on areas or situations that the platform has identified as being important to the relationship based on what the partners are engaging in the mobile application. For example, if a partner watches a video or attends a live webinar, the platform will create a dynamic question based on getting a read on this area. If the partner is sending gifts or has completed a activity like First Step, the questions will be directed to that area.

In some embodiments, the machine learning and AI algorithms on the remote server can be configured to have a partner go deeper into an area they have reflected or identified as causing a challenge, and can be followed by a detailed set of onboarding questions for that area. For example, if someone has in-law issues, then the questions can be directed to a more detailed challenge they have with this exact situation.

In some embodiments, the platform will store this and, via dynamic content, provide an invite to the partner to watch a video, attend a live event, take part in a First Step activity with these areas and selection. In some embodiments, the dynamic content is based on the current strength of the relationship, as well as holidays and life events (e.g., Valentine's day, Mother's day, Father's day, birthdays, anniversaries, new jobs, interventions, starting a self-help program, etc.). This type of monitoring is extremely important as it advantageously enables the relationship indices to be continually updated.

Emotional Bank Account. This method is a motivation tool that encourages Partner A to point out the little things Partner B does (e.g., kudos or encouragements) and then it prompts Partner B to reciprocate and send Partner A points creating a loop and promoting healthy relationship principles, as shown in 6A-6F.

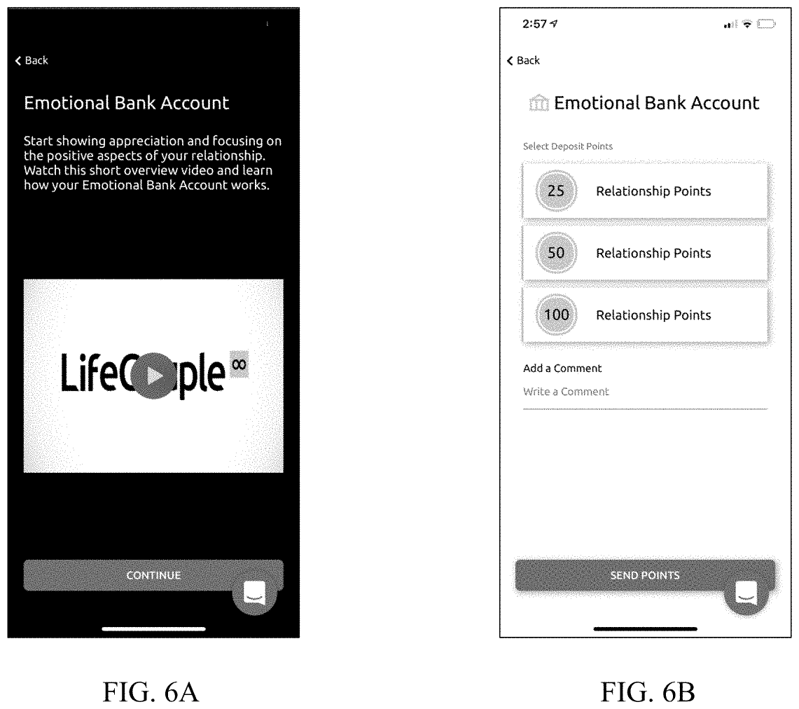

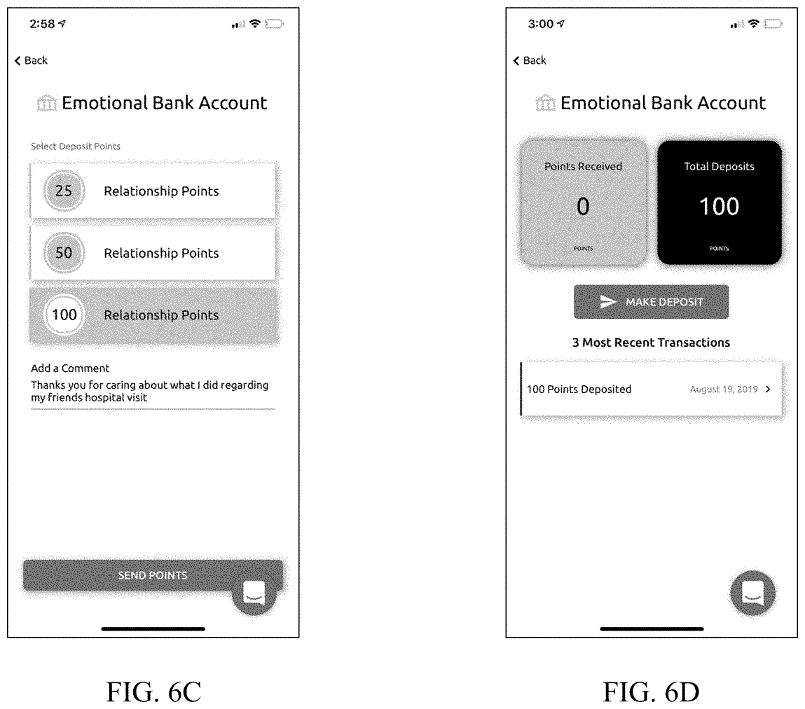

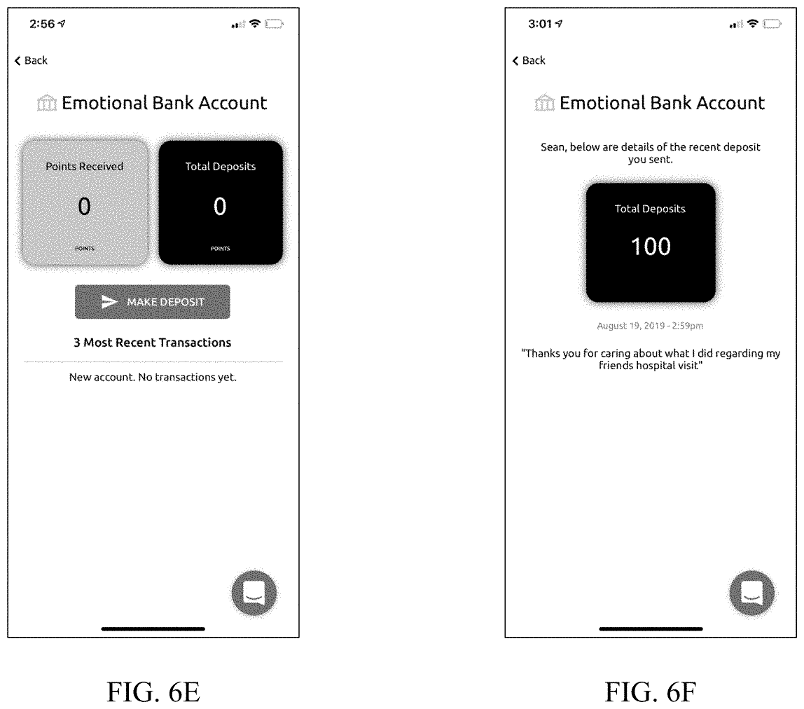

In some embodiments, Partner A is instructed to watch a video and is told how the tools works. They then go to screen that allows them to select points based on what they judge is the value of the action (e.g., activities may be selected at the 25, 50 and 100 point level, as shown in FIG. 6B), and are required to input a description of the action and why they are giving points (e.g., FIG. 6C). If Partner B is not synced then the platform requires you to invite your partner. Once synced the two partners see how many points they deposited and how many they received (e.g., in direct analogy to a bank account). The platform keeps track of the transactions (FIGS. 6D-6F) and allows both partners to have an expanded view of the relationship, and thus be able to keep track on with positive therapy principles.

In some embodiments, and based on the interaction of the partners and how many points they give each other, the remote server may periodically invite each partner to gauge their relationship health, which updates the relationship indexes. In other embodiments, and after a certain number of points are accumulated, third-party offers and incentives may be unlocked in an effort to promote this positive behavior in the relationship.

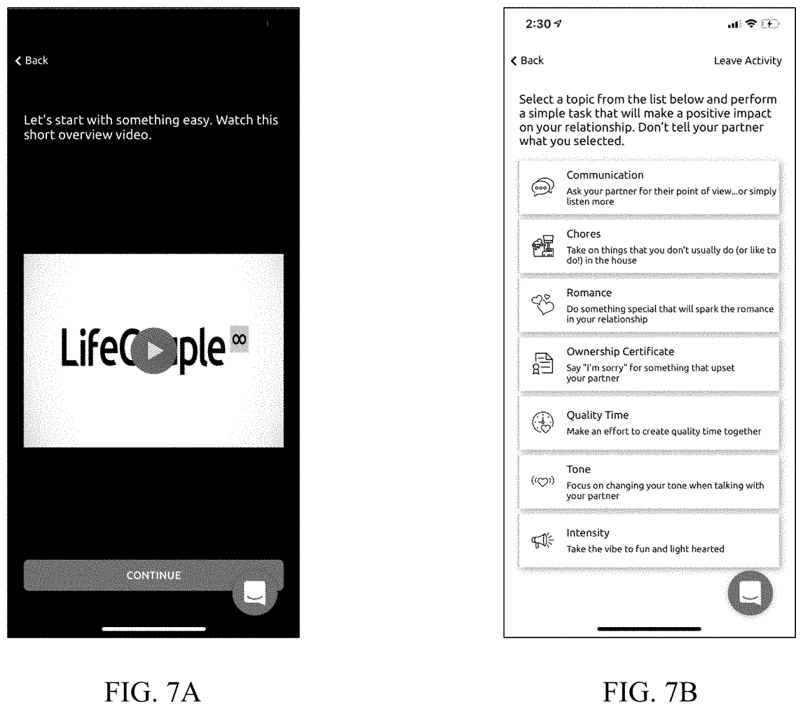

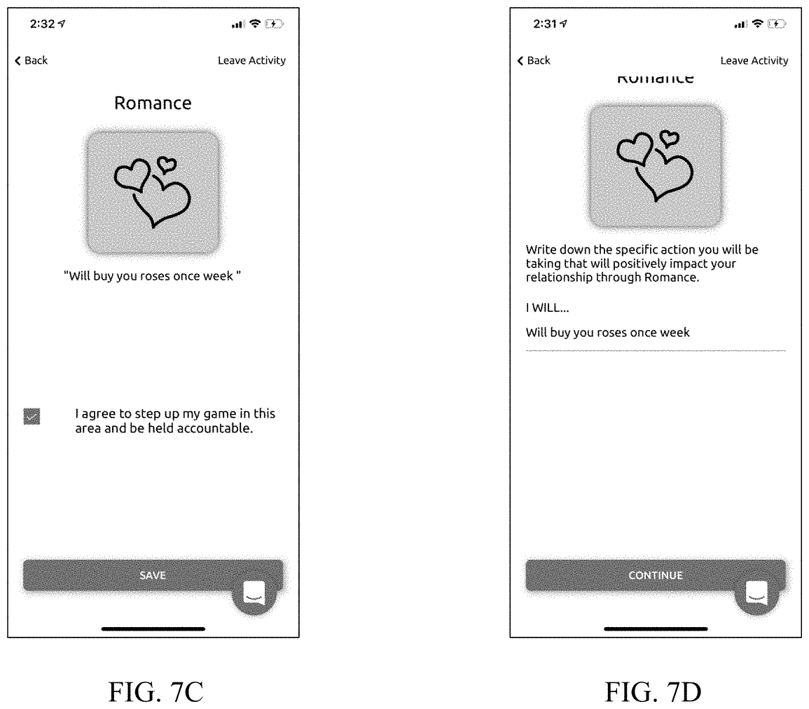

First Step. In this method, shown in FIGS. 7A-7D, Partner A watches a video (e.g., FIG. 7A) and reads about the purpose and goal of the activity. Then, they get a drop down of areas they can select and work on (e.g., FIG. 7B), and must also describe the specific activity that Partner A is encouraged to perform and agree to be accountable for (e.g., make dinner, positive interaction with in-laws). Partner A must then check a box to agree that they are going to follow through on this activity (e.g., FIG. 7C), and gets reminder and encouragement emails and text messages to complete the activity. In this method, Partner A must invite Partner B in order for the efficacy of the First Step method to be realized.

In some embodiments, the platform will subsequently ask Partner B (e.g., in a week) to guess the area that Partner A selected, and the platform will determine whether Partner B was correctly able to identify the area. If Partner B got it correct then the platform will give them points, which are tracked and may be used to in gamification aspects (e.g., move up levels, receive third-party incentives, etc.).

In some embodiments, the First Step activity may be initiated and performed for various areas in a relationship, including finance issues, dealing with the in-laws, etc.

In some embodiments, the First Step activity can be used repeatedly so that Partners A and B can continue to work on their relationship, gain points, etc.

In some embodiments, engaging in several First Step activities is followed up by a request to perform the Relationship Pulse Check-In activity, which will update the relationship indices, and compare the results to those from the initial onboarding process.

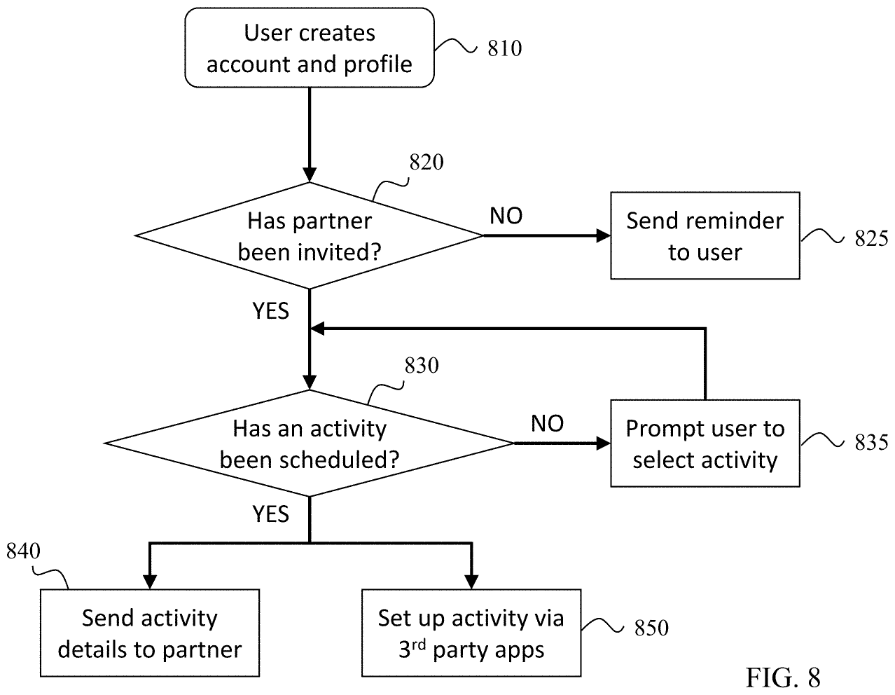

FIG. 8 shows an example of a method performed by the remote server (e.g., 130 in FIG. 1) that encourages a user to set up an activity in order to strength the user's relationship (e.g., as part of the First Step or Emotional Bank Account activities). As shown therein, the method begins with the user creating an account and profile 810. The remote server checks whether the user has invited their partner to install software associated with embodiments of the disclosed technology 820, and if the user has not, a reminder to send an invitation may be sent to the user 825. In some embodiments, the reminder to the user may be re-transmitted periodically.

Based on the relationship metrics and status between the user and partner, the remote server may check whether an activity has been scheduled 830. If an activity has not been scheduled, the user is prompted to select an activity 835. Once the activity has been selected by the user, the remote server can send the activity details to the partner 840 and organize the activity using, for example, 3rd party application 850.

### 3.1 Veracity Monitoring

In some embodiments, the above activities may be performed in conjunction with the video and biometric functionalities of the mobile device, which will monitor the users while they take surveys, respond to questions, set up activities, etc. This will advantageously allow the platform to gauge the veracity or truthfulness of the users.

For an example, hand movements when interacting the mobile application can be tracked since dishonest interactions often take longer and involve more hand movement than honest ones. For another example, truthfulness may be determined by measuring subtle changes in a person's eyes, such as pupil dilation and blink rate. For yet another example, the video camera on the device can be used to capture changes in blood flow in a person's face. The camera may capture light in the visible, infrared, or ultraviolet spectrums for use in determining veracity. As another example, pulse rate may be monitored using a fitness tracker or data on the mobile device obtained from a fitness tracker and correlated with interactions on the mobile application.

In some embodiments, the biometric information collected that is used for veracity monitoring can also be used to track fitness levels and physical activity engagement (e.g., heart rate). In an example, a user's fitness and activity level is monitored and correlated with their relationship scores and indices. A positive correlation may be followed by a suggested activity that promotes this behavior.

### 3.2 Time-Together Metric

In some embodiments, the relationship indices may be updated based on a time-together metric that leverages the basic functionality of the mobile device. For an example, the wireless capabilities or location capabilities (e.g. GPS) of the device may be used to determine whether Partners A and B are in proximity of each other, but actual use on the phone can be used to gauge whether they are actually interacting with each other or simply doing their own thing in the same room at the same time. If the platform determines that 10s of minutes have been spent with no interaction, a push email or text message may be delivered to both partners to suggest an activity for engagement.

For another example, the virtual interactions between the partners (e.g., Facetime, messaging or calling each other) can be tracked and used to update the relationship indices, suggest activities and/or provide data to third-party mediators.

As another example, the location capabilities can be used to measure work-life balance by comparing the time at work or work-related activities to time at home and home-related activities. A user may be prompted to categorize certain locations (e.g. public park) as work or home to help measure work-life balance. Time commuting to and from work may also be measured as this time has been found to decrease happiness.

### 3.3 Photo Analytics

In some embodiments, the platform may access the photo archive in a user's mobile device and use contents of the digital photos to adjust the relationship indices, suggest activities and/or provide data to third-party mediators. For an example, the inclusion of the other partner (or other family members, e.g., kids, parents) in photographs on a partner's phone may be indicative of the strength of the relationship. For another example, similar photos or photos that were taken at the same place at the same time may be indicative of the time that the partners are spending with each other (e.g., on a common holiday, as compared to hanging out to their own friends with their significant other) and can be used to adjust the time-together metric to show the time spent together is more memorable.

### 3.4 Data Ownership/Control

In some embodiments, the platform can be configured to update the user interface as well as the activities and tools when a particular relationship ends. For an example, if Employee A leaves the company, the platform for the other employees will be updated to reflect the departure of Employee A, as will be platform for Employee A. In addition to the platforms being updated, both Employee A and the other employees with be provided with any data that the company requires be deleted, and they can then determine whether (and if) any of that data should be stored on a personal cloud drive. Embodiments of disclosed technology may be configured to provide users with ownership of their data and the ability to delete some or all of their data from the mobile application or web/analysis interface of others. Some data, for example anonymized data, can in some examples be maintained by others for use research purposes (e.g., cross-sectional and longitudinal studies) after it has been anonymized.

### 3.5 Transitioning to the Next Phase in Life

In some embodiments, the mobile application, in conjunction with the ML and AI capabilities of the remote server and third-party mediation, can be used to assist with a user transitioning from one phase of his/her life to the next. The data collected from a user can be compared to data from a variety of groups the user is part of, to determine what factors should be specifically addressed when a major life change occurs.

In an example, if a relationship has ended in a separation or divorce, the platform may dynamically encourage the user to maintain their sense of self, personal state of happiness, etc. In another example, an athlete that is retiring may be guided to focus on other avenues that are now options for the next phase of their life. In yet another example, an employee who is switching jobs may be encouraged to not burn bridges at their former employer, and continue to maintain contacts and seek networking opportunities.

### 4. Embodiments of the Web/Analysis Interface

Embodiments of the disclosed technology have the ability to integrate with therapists, HR departments, parents, team personal heads, etc. In general, the users on the mobile application (120 in FIG. 1) can leverage a third-party mediator (140 in FIG. 1) to help the users remain accountable to each other and their goals.

In an example, the remote server (via the mobile app) can offer the dashboards and functions so the practitioner or the machine learning via AI can make a suggestion on what activities can be automatically pushed the mobile application. The mobile application users will get automatic messaging and reminders to complete and engage in the content, and when the user comes back into the therapist office the results are available to see progress and create a treatment plan.

In some embodiments, the tracking of weeks, months and years provide the therapist and their clients (or patients) with the ability to set goals and work towards positive empowerment.

In some embodiments, the platform may generate a summary of results for the interactions between the partners and provide that to the therapist so that specific issues can be addressed on the first day of therapy (instead of the more traditional intake appointment which may take several sessions, and is must less effective).

Embodiments of the disclosed technology further provide methods of reporting progress back to insurance companies to ensure payments on the practitioners side are paid in a timely fashion. The described embodiments can advantageously change the mental health space as it allows the therapist to have valuable insight on the state of the relationship and make the first few sessions effective (which are typically not since the therapist is still onboarding the clients).

In another example, embodiments of the disclosed technology enable employees to be completely confidential in their candid feedback, which ensures employee satisfaction in various departments (e.g., warehousing, marketing, accounting, etc.) can be gauged in real-time by the HR department and/or senior leadership. That is, the described dashboard can offer a source of truth vs the old fashion other assessments that have no mobile app integration.

In yet another example, and from the standpoint of team sports or athletes, embodiments of the disclosed technology can gauge many key aspects and see how a team personal can offer team building and per player activities and tools, thereby protecting the substantial investment in player salaries. That is, the embodiments described herein can be used to quantify the notions of team unity and locker room culture, which correlate to winning teams.

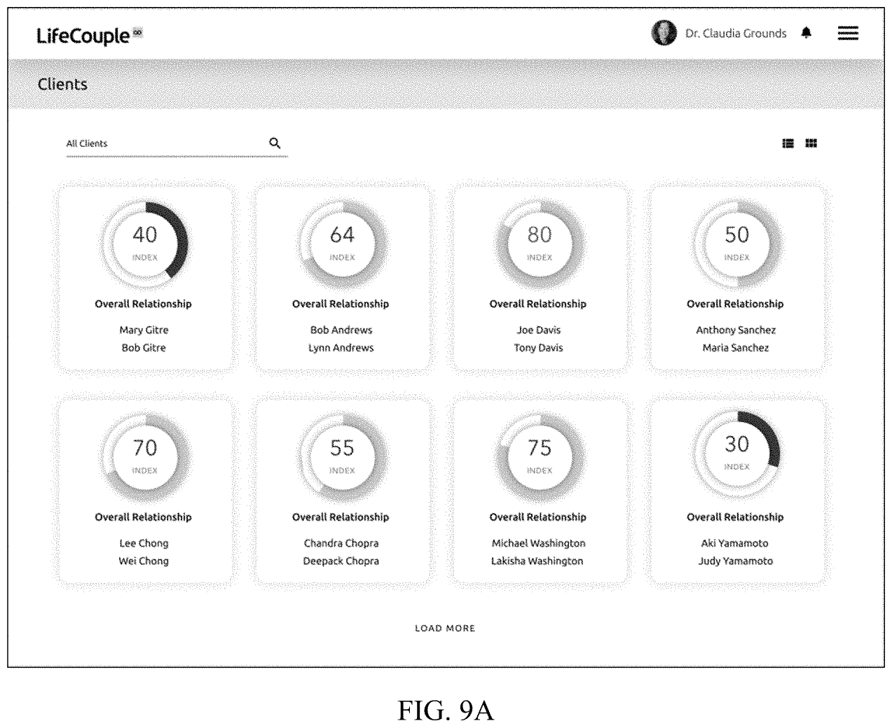

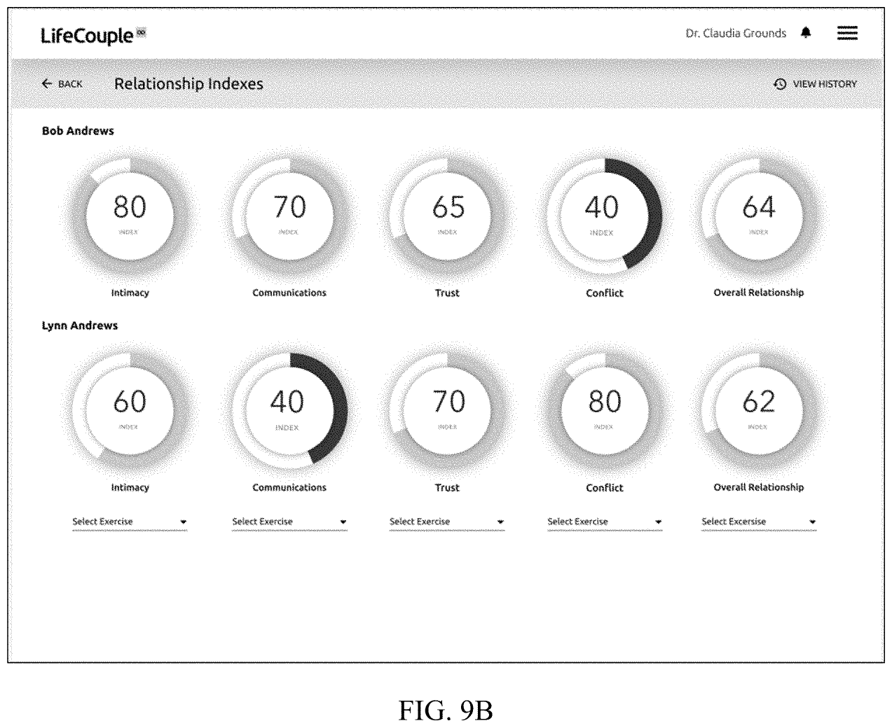

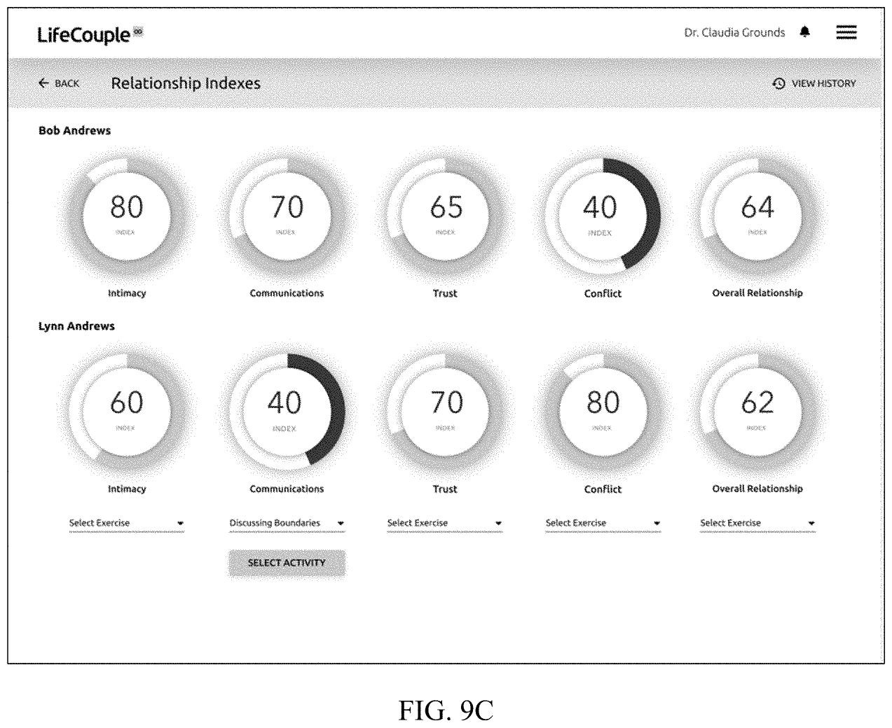

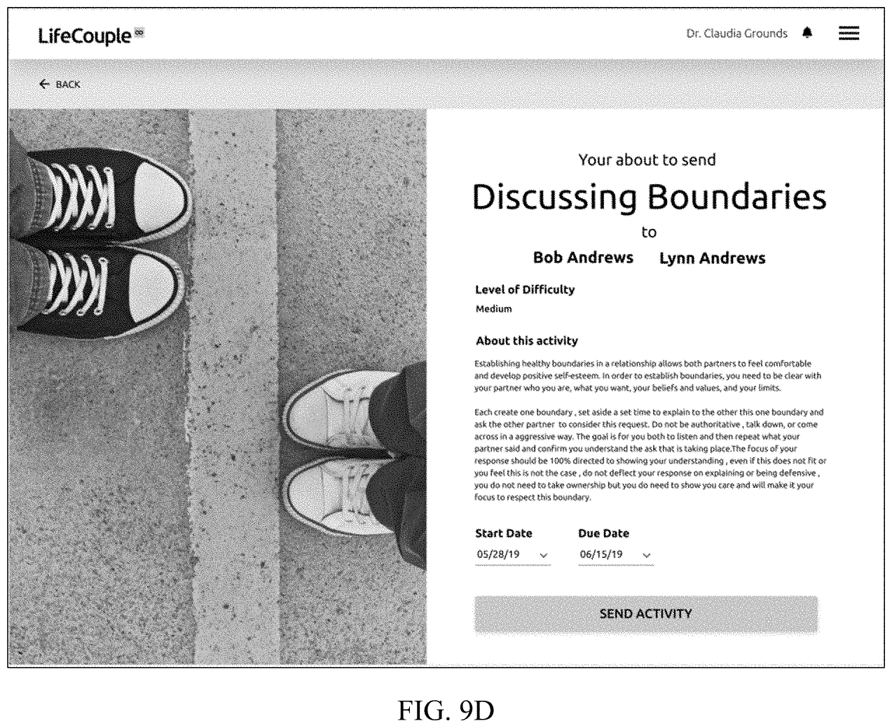

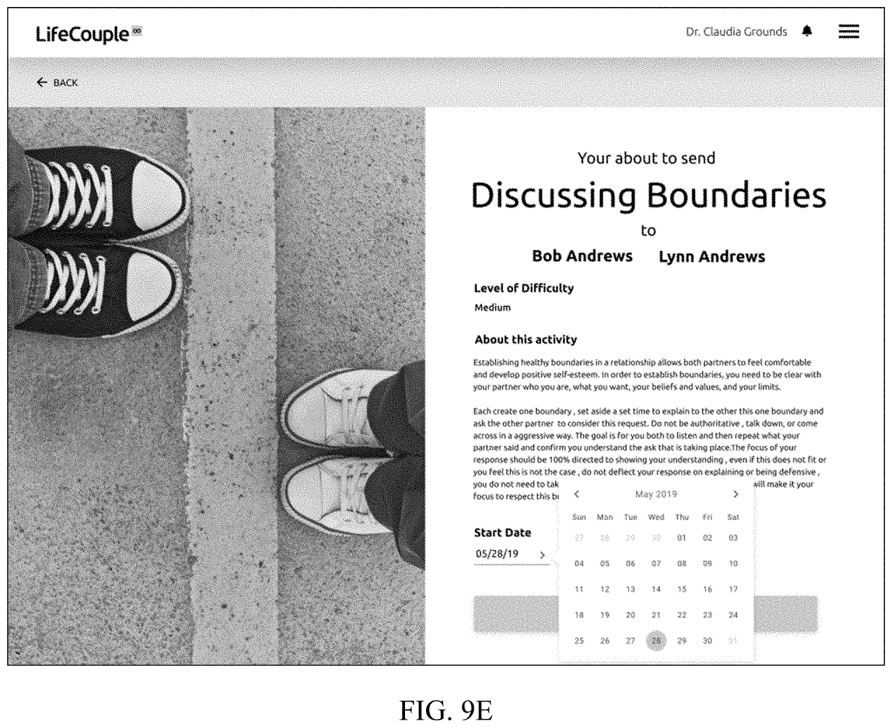

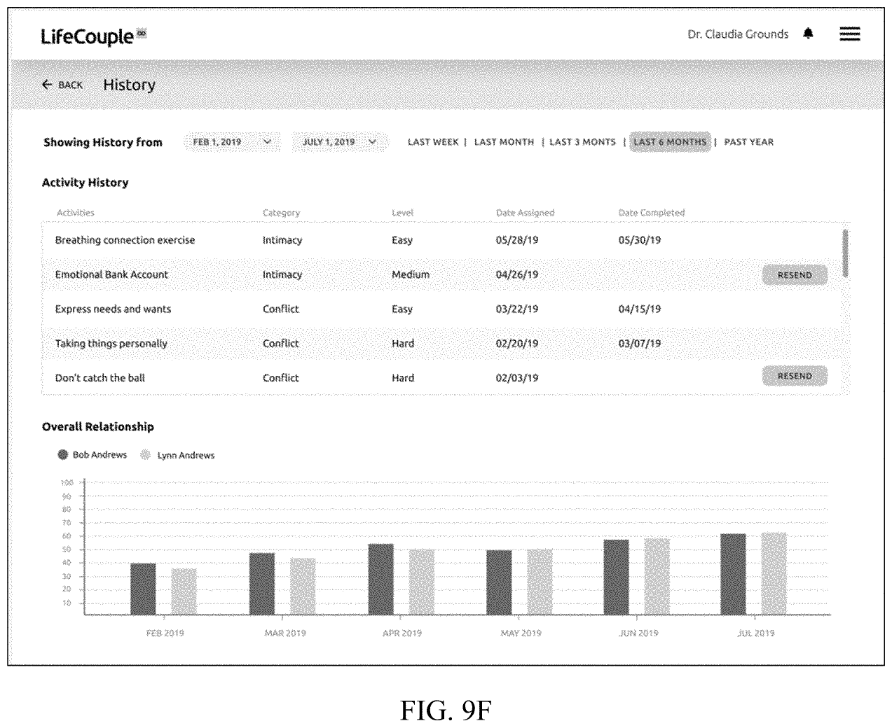

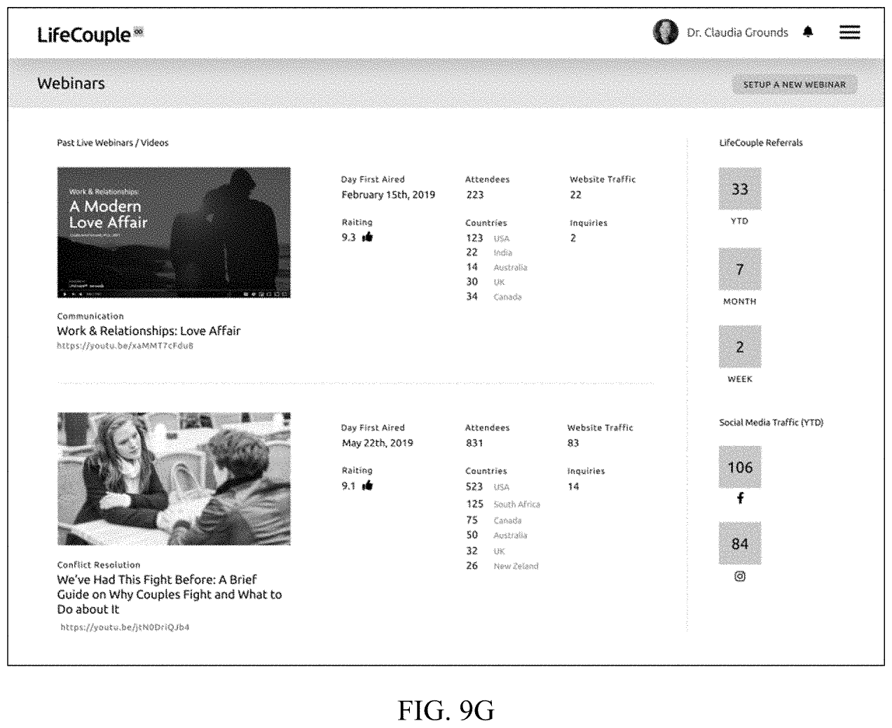

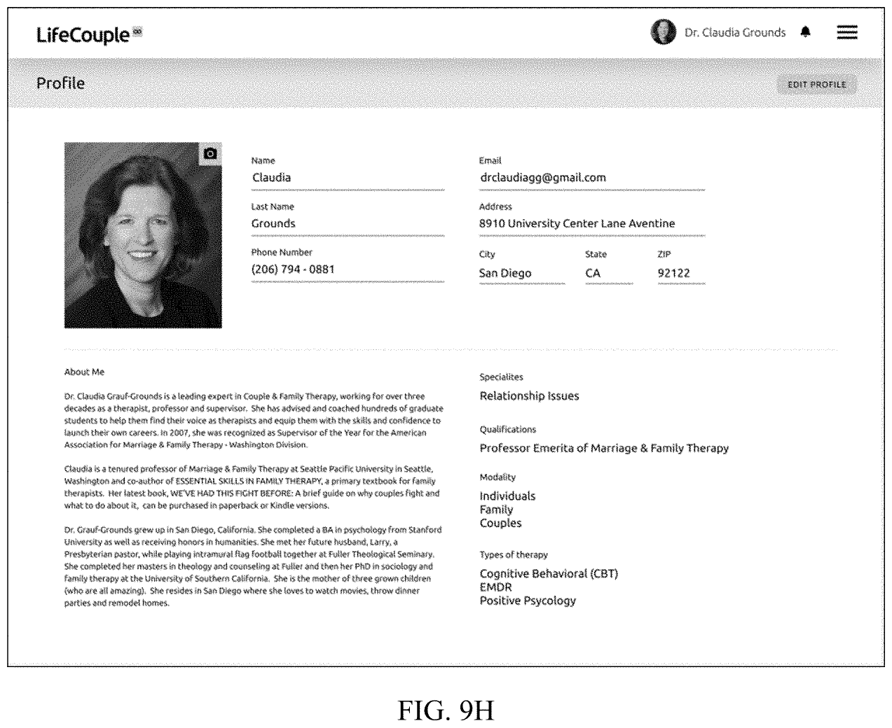

In some embodiments, the integration with third-part mediators (e.g., the therapist) also offers ways for the therapist to use the dashboard and track various aspects and behaviors of the clients who are using the mobile application. An example of the dashboard, and its exemplary functionality is shown in FIGS. 9A-9H. As shown in FIG. 9A, the therapist can view the overall (or family) score of each couple that he/she is working with. The individual indexes for each partner of a couple can be viewed (e.g., FIG. 9B) and a an activity selected to improve a particular facet of the relationship (e.g., FIG. 9C).

In the example shown in FIGS. 9D and 9E, the activity and start date for that activity (e.g., “Discussing Boundaries”) can be selected and sent to a particular couple. This activity, along with the previous assigned activities (and their corresponding status) can be viewed by the therapist (e.g., FIG. 9F). In some embodiments, the platform can integrate commercially available videos to address specific relationship issues, and make these videos available for the users (e.g., FIG. 9G).

In an example, a therapist may offer a live event using embodiments of the disclosed technology and based on the integration dashboard, they can see what audience they are obtaining and then what traffic is being driven to their personal website or social media account.

In yet another example, the web/analysis interface (e.g., the dashboard) can be used to track leads and businesses that a client may have developed through the mobile application.

In yet another example, clients may be asked several questions (e.g., as part of the onboarding process), which may be used to find a potential therapist (e.g., FIG. 9H). That is, the answers to the questions can be used to employ a specific psychological or therapeutic method (e.g., the John Gottman method, the Sue Johnson method) that will be most advantageous to the clients and their efforts to foster their relationship dynamics and situation.

In yet another example, users in the platform can create a customized summary of their relationship data, and can select any or all of it to be shared.

### 4.1 Moderated Messaging System

As described in the above embodiments, Partner A and Partner B are monitored separately; each have individual metrics but there is also a metric for the relationship (or family score when applicable). Thus, embodiments of the disclosed technology may be configured to enable moderated or curated communications between the two partners. For example, and as shown in FIG. 3B, some information might be shared only with the therapist (or the other partner), whereas some information is shared with both the therapist and the other partner.

Fostering and building a relationship may not always be helped by constant direct communication between partners. In contrast to conventional chat programs, the platform may be configured to both create an asymmetric communication situation (e.g., some messages not being sent to the other partner, but only to the therapist) as well as modify the content of one or more communications so it remains either neutral or positive.

In contrast to conventional therapy sessions which involve a therapist speaking with either both partners or one partner in the explicit absence of the other partners, embodiments of the disclosed technology may be configured so that the therapist may advantageously communicate with both partners through the platform, and simultaneously provide individual messaging to one of the partners, which could be extremely helpful in resolving conflicts between partners in certain contentious areas of the relationship.

### 5. Example Method and Embodiments of the Disclosed Technology

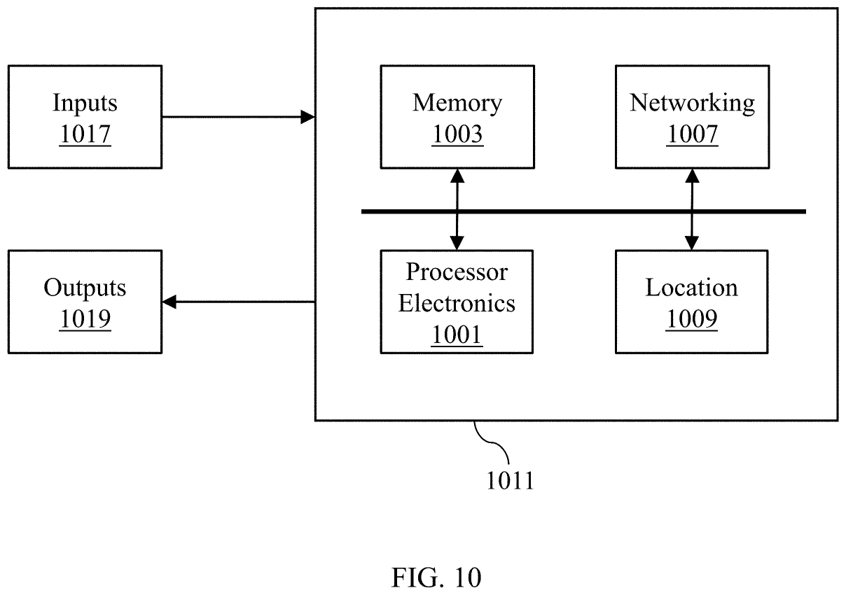

FIG. 10 is a block diagram representation of a portion of a device, in accordance with some embodiments of the presently disclosed technology. A device 1011 (e.g., which may correspond to one of the mobile applications 120, the web/analysis interface 140 or the remote server 130 shown in FIG. 1) can include processor electronics 1001 such as a microprocessor that implements one or more of the techniques presented in this document, including tracking one or more metrics associated with the relationship. Device 1011 can include one or more memories 1003 configured to store information such as data and/or instructions. Device 1011 can further include networking functionality 1007 and location capabilities 1009. In some embodiments, the device may be coupled to input devices 1017 (e.g., a user interface and/or camera) and output devices 1019 (e.g., a display and/or speaker). In some embodiments, at least some of the disclosed techniques, modules or functions are implemented using the device 1011.

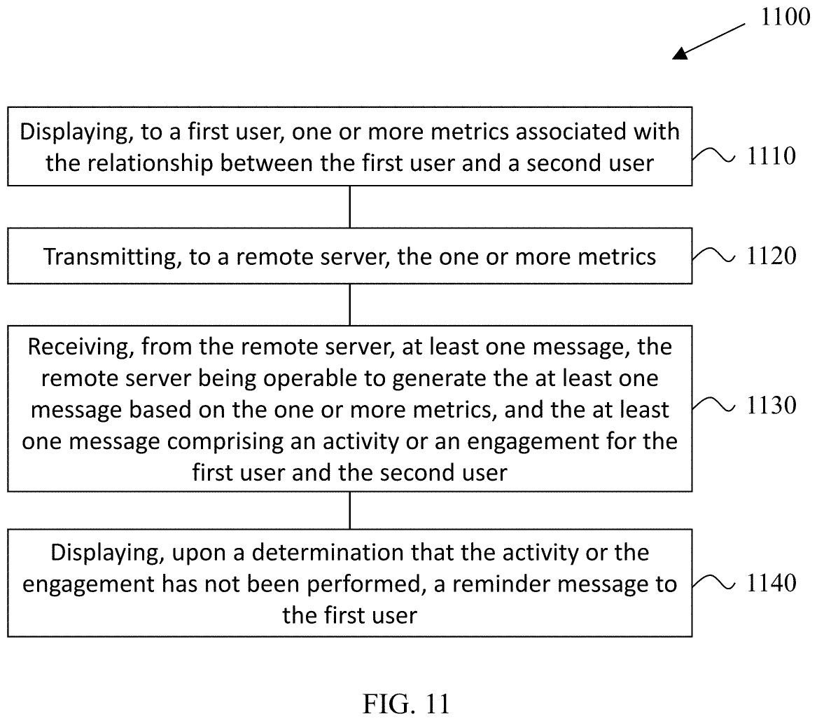

The examples and embodiments described above can be used to implement at least the following methods described in FIGS. 11-13.

FIG. 11 is a flowchart illustrating an example method 1100 for monitoring a relationship. Method 1100 includes, at operation 1110, displaying, to a first user, one or more metrics associated with the relationship between the first user and a second user.

Method 1100 includes, at operation 1120, transmitting, to a remote server, the one or more metrics.

Method 1100 includes, at operation 1130, receiving, from the remote server, at least one message, the remote server being operable to generate the at least one message based on the one or more metrics, and the at least one message comprising an activity or an engagement for the first user and the second user.

Method 1100 includes, at operation 1140, displaying, upon a determination that the activity or the engagement has not been performed, a reminder message to the first user.

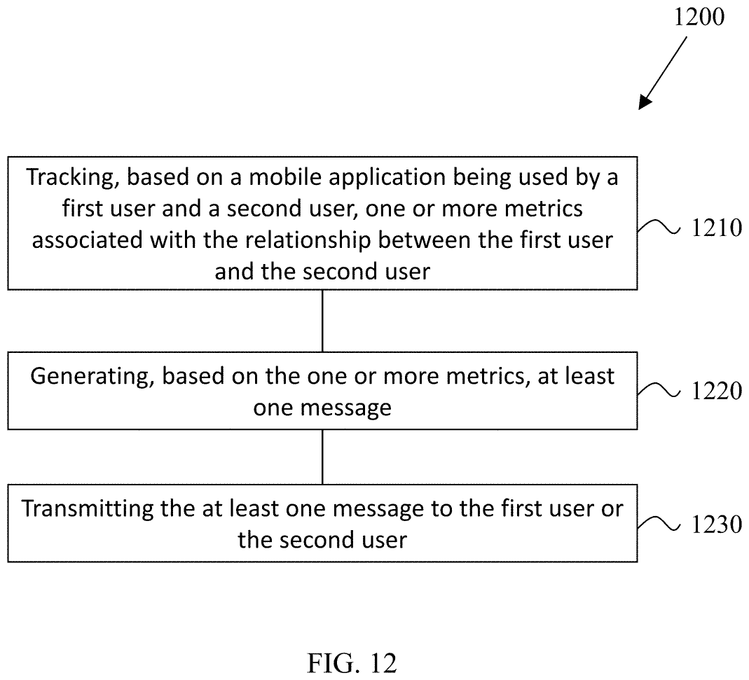

FIG. 12 is a flowchart illustrating an example method 1200 for monitoring a relationship. Method 1200 includes, at operation 1210, tracking, based on a mobile application being used by a first user and a second user, one or more metrics associated with the relationship between the first user and the second user.

Method 1200 includes, at operation 1220, generating, based on the one or more metrics, at least one message.

Method 1200 includes, at operation 1230, transmitting the at least one message to the first user or the second user.

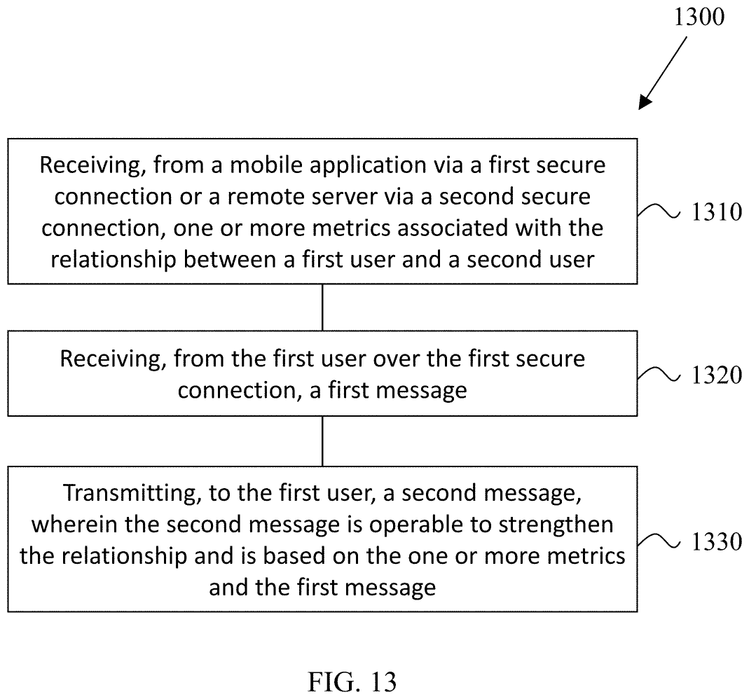

FIG. 13 is a flowchart illustrating an example method 1300 for monitoring a relationship. Method 1300 includes, at operation 1310, receiving, from a mobile application via a first secure connection or a remote server via a second secure connection, one or more metrics associated with the relationship between a first user and a second user.

Method 1300 includes, at operation 1320, receiving, from the first user over the first secure connection, a first message.

Method 1300 includes, at operation 1330, transmitting, to the first user, a second message, wherein the second message is operable to strengthen the relationship and is based on the one or more metrics and the first message.

Embodiments of the disclosed technology provide the following technical solutions that are directed to tracking, measuring, and advising users' interactions with their partners in different aspects of their relationship, thereby providing a sense of accountability for all participants, and strengthening the relationship.

1. A method for monitoring a relationship, comprising displaying, to a first user, one or more metrics associated with the relationship between the first user and a second user; transmitting, to a remote server, the one or more metrics; receiving, from the remote server, at least one message, wherein the remote server is operable to generate the at least one message based on the one or more metrics, and wherein the at least one message comprises an activity or an engagement for the first user and the second user; and displaying, upon a determination that the activity or the engagement has not been performed, a reminder message to the first user.

2. The method of solution 1, wherein the one or more metrics comprise an intimacy metric, a communication metric, a trust metric, a conflict metric, a time-together metric, and an overall relationship metric.

3. The method of solution 2, the one or more metrics are generated based on feedback from the first user and the second user.

4. The method of solution 1, wherein the activity or the engagement comprises dealing with a difficult relationship topic, gauging a current health of the relationship, and encouraging a performance of an action that is meaningful to either the first user or the second user.

5. The method of solution 4, wherein the performance of the action by the first user is used to incentivize the second user to perform a reciprocal action.

6. The method of solution 1, wherein the reminder message is re-sent periodically upon a determination that the activity or the engagement still has not been performed.

7. The method of solution 1, further comprising tracking a performance of the activity or the engagement; and updating, based on the tracking, the one or more metrics.

8. A method for monitoring a relationship, comprising tracking, based on a mobile application being used by a first user and a second user, one or more metrics associated with the relationship between the first user and the second user; generating, based on the one or more metrics, at least one message; and transmitting the at least one message to the first user or the second user.

9. The method of solution 8, wherein the one or more metrics comprise an intimacy metric, a communication metric, a trust metric, a conflict metric, and an overall relationship metric.

10. The method of solution 9, further comprising normalizing, based on one or more predetermined factors, the overall relationship metric to generate a normalized overall relationship metric, wherein the normalizing comprises adjusting the overall relationship metric based on inherent limitations associated with the one or more predetermined factors.

11. The method of solution 10, wherein the one or more predetermined factors comprises at least one of an age, a salary, or a zip code of a residence of the first user or the second user.

12. The method of solution 10, wherein the first user and the second user are in a marital relationship, or members of a sports team, or employees of a company.

13. The method of solution 8, wherein the relationship is a marital relationship, and wherein the one or more metrics comprise a score based on the Locke-Wallace Marital Adjustment Test (MAT), the Quality of Marriage Index (QMI), the Relationship Assessment Scale (RAS), or the Kansas Marital Satisfaction Scale (KMSS).

14. The method of solution 8, wherein the mobile application comprises a photo acquisition and storage functionality, and wherein at least one of the one or more metrics is updated based on the photo acquisition and storage functionality.

15. A method for monitoring a relationship, comprising receiving, from a mobile application via a first secure connection or a remote server via a second secure connection, one or more metrics associated with the relationship between a first user and a second user; receiving, from the first user over the first secure connection, a first message; and transmitting, to the first user, a second message, wherein the second message is operable to strengthen the relationship and is based on the one or more metrics and the first message.

16. The method of solution 15, wherein the second message is advise from a therapist.

17. The method of solution 16, wherein the second message is configured to be accessible only to the first user and not to the second user.

18. The method of solution 16, wherein the second message comprises electronic protected health information (ePHI), and wherein transmitting the second message is in compliance with the Health Insurance Portability and Accountability Act (HIPAA).

19. The method of solution 15, wherein the one or more metrics comprise an intimacy metric, a communication metric, a trust metric, a conflict metric, and an overall relationship metric.

20. The method of solution 15, wherein the second message is further based on a history of the relationship over a predetermined time duration.

Implementations of the subject matter and the functional operations described in this patent document can be implemented in various systems, digital electronic circuitry, or in computer software, firmware, or hardware, including the structures disclosed in this specification and their structural equivalents, or in combinations of one or more of them. Implementations of the subject matter described in this specification can be implemented as one or more computer program products, i.e., one or more modules of computer program instructions encoded on a tangible and non-transitory computer readable medium for execution by, or to control the operation of, data processing apparatus. The computer readable medium can be a machine-readable storage device, a machine-readable storage substrate, a memory device, a composition of matter effecting a machine-readable propagated signal, or a combination of one or more of them. The term “data processing unit” or “data processing apparatus” encompasses all apparatus, devices, and machines for processing data, including by way of example a programmable processor, a computer, or multiple processors or computers. The apparatus can include, in addition to hardware, code that creates an execution environment for the computer program in question, e.g., code that constitutes processor firmware, a protocol stack, a database management system, an operating system, or a combination of one or more of them.

A computer program (also known as a program, software, software application, script, or code) can be written in any form of programming language, including compiled or interpreted languages, and it can be deployed in any form, including as a stand-alone program or as a module, component, subroutine, or other unit suitable for use in a computing environment. A computer program does not necessarily correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform functions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Computer readable media suitable for storing computer program instructions and data include all forms of nonvolatile memory, media and memory devices, including by way of example semiconductor memory devices, e.g., EPROM, EEPROM, and flash memory devices. The processor and the memory can be supplemented by, or incorporated in, special purpose logic circuitry.

While this patent document contains many specifics, these should not be construed as limitations on the scope of any invention or of what may be claimed, but rather as descriptions of features that may be specific to particular embodiments of particular inventions. Certain features that are described in this patent document in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. Moreover, the separation of various system components in the embodiments described in this patent document should not be understood as requiring such separation in all embodiments.

Only a few implementations and examples are described and other implementations, enhancements and variations can be made based on what is described and illustrated in this patent document.

What is claimed is:

1. A method for monitoring a relationship, comprising:
displaying, to a first user, one or more metrics associated with the relationship between the first user and a second user;
transmitting, to a remote server, the one or more metrics;
receiving, from the remote server, at least one message, wherein the remote server is operable to generate the at least one message based on the one or more metrics, and wherein the at least one message comprises an activity or an engagement for the first user and the second user; and
displaying, upon a determination that the activity or the engagement has not been performed, a reminder message to the first user.

2. The method of claim 1, wherein the one or more metrics comprise an intimacy metric, a communication metric, a trust metric, a conflict metric, a time-together metric, and an overall relationship metric.

3. The method of claim 2, the one or more metrics are generated based on feedback from the first user and the second user.

4. The method of claim 1, wherein the activity or the engagement comprises dealing with a difficult relationship topic, gauging a current health of the relationship, and encouraging a performance of an action that is meaningful to either the first user or the second user.

5. The method of claim 4, wherein the performance of the action by the first user is used to incentivize the second user to perform a reciprocal action.

6. The method of claim 1, wherein the reminder message is re-sent periodically upon a determination that the activity or the engagement still has not been performed.

7. The method of claim 1, further comprising:
tracking a performance of the activity or the engagement; and
updating, based on the tracking, the one or more metrics.

8. A method for monitoring a relationship, comprising:
tracking, based on a mobile application being used by a first user and a second user, one or more metrics associated with the relationship between the first user and the second user;
generating, based on the one or more metrics, at least one message; and
transmitting the at least one message to the first user or the second user.

9. The method of claim 8, wherein the one or more metrics comprise an intimacy metric, a communication metric, a trust metric, a conflict metric, and an overall relationship metric.

10. The method of claim 9, further comprising:
normalizing, based on one or more predetermined factors, the overall relationship metric to generate a normalized overall relationship metric,
wherein the normalizing comprises adjusting the overall relationship metric based on inherent limitations associated with the one or more predetermined factors.

11. The method of claim 10, wherein the one or more predetermined factors comprises at least one of an age, a salary, or a zip code of a residence of the first user or the second user.

12. The method of claim 10, wherein the first user and the second user are in a marital relationship, or members of a sports team, or employees of a company.

13. The method of claim 8, wherein the relationship is a marital relationship, and wherein the one or more metrics comprise a score based on the Locke-Wallace Marital Adjustment Test (MAT), the Quality of Marriage Index (QMI), the Relationship Assessment Scale (RAS), or the Kansas Marital Satisfaction Scale (KMSS).

14. The method of claim 8, wherein the mobile application comprises a photo acquisition and storage functionality, and wherein at least one of the one or more metrics is updated based on the photo acquisition and storage functionality.

15. A method for monitoring a relationship, comprising:
receiving, from a mobile application via a first secure connection or a remote server via a second secure connection, one or more metrics associated with the relationship between a first user and a second user;
receiving, from the first user over the first secure connection, a first message; and
transmitting, to the first user, a second message, wherein the second message is operable to strengthen the relationship and is based on the one or more metrics and the first message.

16. The method of claim 15, wherein the second message is advise from a therapist.

17. The method of claim 16, wherein the second message is configured to be accessible only to the first user and not to the second user.

18. The method of claim 16, wherein the second message comprises electronic protected health information (ePHI), and wherein transmitting the second message is in compliance with the Health Insurance Portability and Accountability Act (HIPAA).

19. The method of claim 15, wherein the one or more metrics comprise an intimacy metric, a communication metric, a trust metric, a conflict metric, and an overall relationship metric.

20. The method of claim 15, wherein the second message is further based on a history of the relationship over a predetermined time duration.
